(19) United States
(12) Patent Application Publication
(10) Pub. No.: US 2015/0351632 A1
(43) Pub. Date: Dec. 10, 2015

(54) SYSTEMS AND METHODS FOR TWO-WAY COMMUNICATION FOR TELEMETRY DEVICE

(71) Applicant: Mindray DS USA, Inc., Mahwah, NJ (US)

(72) Inventor: Kenneth J. Fuchs, Wayland, MA (US)

(21) Appl. No.: 14/298,664

(22) Filed: Jun. 6, 2014

(51) Int. Cl. A61B 5/00; A61B 5/1455; A61B 5/0402

(52) U.S. Cl. CPC A61B 5/0002; A61B 5/0402; A61B 5/14551; A61B 5/7465

(57) ABSTRACT

A portable telemetry device includes a measurement component, a wireless radio, a data communication component, and a voice communication component. The measurement component is configured to receive, from at least one sensor, physiological data representative of a physiological condition of a patient. The wireless radio is configured to wirelessly send and receive radio signals. The data communication component is configured to transmit the physiological data to a monitoring system using the wireless radio. The voice communication component is configured to provide, using the wireless radio, two-way voice communication between the portable telemetry device and medical staff at the monitoring system.

### TECHNICAL FIELD

The present disclosure relates to medical monitoring and more particularly relates to systems, methods, and devices for two-way communication for medical telemetry.

### BRIEF DESCRIPTION OF THE DRAWINGS

FIG. 1 is a schematic block diagram illustrating a telemetry system, according to one embodiment.

FIG. 2 is a schematic block diagram illustrating a portable telemetry device, according to one embodiment.

FIG. 3 is a schematic block diagram illustrating a monitoring system, according to one embodiment.

FIG. 4 is a schematic diagram illustrating a portable telemetry device in communication with a monitoring system, according to one embodiment.

FIG. 5 is a schematic flow chart diagram illustrating a method for wireless telemetry, according to one embodiment.

FIG. 6 is a schematic flow chart diagram illustrating another method for wireless telemetry, according to another embodiment.

### DETAILED DESCRIPTION OF PREFERRED EMBODIMENTS

Modern medical technology practice makes extensive use of electronic monitoring of vital signs and other physiological parameters of patients. In some cases, remote monitoring of physiological parameters, or telemetry, is used to allow nurses, doctors, and/or computing devices to determine the health of a patient or detect problems with the patient when the nurse or doctor is not present with the patient. In some cases, wireless telemetry devices worn by a patient, or patient-worn telemetry devices, may allow the patient to move around and/or be easily moved between locations while continuing to monitor the patient's vital signs. For example, some patients benefit from ambulation during a recovery process for physical and/or mental health. Thus, wireless telemetry devices worn by a patient may allow the patient to move about, or be moved about, without pausing or stopping the gathering of physiological parameters and/or without connecting or reconnecting sensor leads or other cables. One example of a portable telemetry device is the Mindray Telepack®.

However, applicants have recognized that patient-worn telemetry devices currently provide little or no ability for communication beyond the gathered physiological parameters. For example, although medical staff may be able to see the physiological parameters, they are unable to determine how the patient is feeling or what may be occurring with the patient without being present with the patient. Some telemetry devices include nurse call buttons, but these only allow the patient to alert someone at a nurse central station. The alert carries no information, and thus the medical staff or responder has no idea what the issue is, especially if there are no visible changes to the patient's vital signs. Furthermore, nurses or other medical staff are unable to contact a patient, except by going to the patient's designated room. If the patient is not there, the medical staff have no way to contact the patient.

The present disclosure proposes systems, methods, and devices for two-way communication between a patient-worn telemetry device or wireless portable telemetry device. According to one embodiment, a portable telemetry device includes a measurement component, a wireless radio, a data communication component, and a voice communication component. The measurement component is configured to receive, from at least one sensor, physiological data representative of a physiological condition of a patient. The wireless radio is configured to wirelessly send and receive radio signals. The data communication component is configured to transmit the physiological data to a monitoring system using the wireless radio. The voice communication component is configured to provide, using the wireless radio, two-way voice communication between the portable telemetry device and medical staff at the monitoring system.

In one embodiment, two-way communication with the portable telemetry device may allow for nurses or other medical staff to gain a better understanding of an issue without being present with a patient. For example, a nurse may be able to determine that certain materials, additional assistance from another nurse, or the like may be needed before visiting the patient at the patient's current location. A patient may be able to report any concerns the patient has, such as feeling faint, nauseated, suddenly tired, or the like while a nurse correlates with changes in vital signs or waveforms. This may allow some issues to be resolved remotely or for medical staff to be forewarned of potential issues with the patient before they show up in patient parameters.

Two-way communication may also allow a nurse to contact a patient, even if the patient is not currently in the patient's room or bed. This may allow the nurse to ask for the patient's location, inform the patient of visitors, ask the patient to return to a room or diagnostic area, or the like. In emergency situations, the ability to ask the patient for the patient's current location may allow medical personnel to come to the patient's aid more quickly.

A detailed description of systems and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

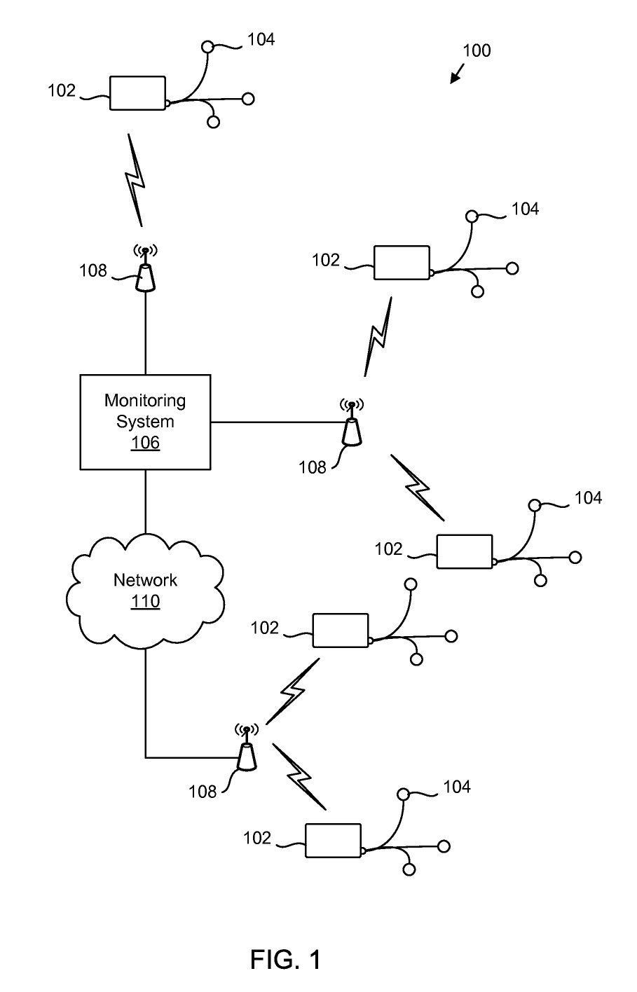

Turning to the figures, FIG. 1 illustrates an embodiment of a telemetry system 100 for medical telemetry. The telemetry system 100 includes a plurality of portable telemetry devices 102 connected to sensors 104. The portable telemetry devices 102 are in communication with a monitoring system 106. The portable telemetry devices 102 communicate with the monitoring system 106 via a plurality of access radios 108 and/or a network 110. In one embodiment, the portable telemetry device 102 includes a telemetry device worn by a patient. For example, the patient may be free to walk or move while wearing the portable telemetry device 102 due to size and capability for wireless communication. The portable telemetry device 102 may be hung on the patient, clipped to clothing, or worn by the patient in any other manner.

The portable telemetry device 102 may include a portable device comprising a housing containing a processor, circuitry, computer readable memory, antenna, radios, and/or the like. The portable telemetry device 102 may be a size such that it can be worn by a patient while allowing the patient to move freely. The telemetry device 102 may include one or more ports for coupling to sensors and receiving signals from the sensors. The portable telemetry device 102 may include a human-machine interface, which may include a display, one or more buttons, and/or indicator lights to allow a human to determine a status of the portable telemetry device 102, enter information, control operation, or otherwise interact with the portable telemetry device 102.

Connected to the telemetry device 102 are a plurality of sensors 104 which may be used to measure patient parameters and/or obtain patient waveforms. For example, the sensors 104 may include one or more electrocardiography (ECG) sensors, a pulse oximetry sensor (e.g., SpO₂), and/or any other sensors. The portable telemetry device 102 may receive signals from the sensors 104 as analog or digital data signals indicating a physiological condition of a patient. The portable telemetry device 102 may transmit physiological data to the monitoring system 106 using a radio. For example, the telemetry device 102 may forward processed or unprocessed sensor data to the monitoring system 106 so that a doctor, nurse, or other medical personnel can monitor a condition of the patient. The portable telemetry device 102 may be configured to provide the physiological data to the monitoring system 106 using access radios 108. For example, the portable telemetry device 102 may be configured to communicate using a frequency or communication standard corresponding to one or more of the access radios 108. In one embodiment, the portable telemetry device 102 is further configured to provide two-way voice communication between medical staff located at the monitoring system 106 and a patient located with the portable telemetry device 102.

The access radios 108 may include any type of radio or access point. For example, the access radios 108 may include a wireless router, base station, or the like. In one embodiment, the access radios 108 are configured to operate within a restricted or licensed frequency. In the United States, the wireless medical telemetry service (WMTS) provides dedicated protected bands which have been allocated for this purpose and which many hospitals prefer to use over the more widely used industrial, scientific, and medical (ISM) radio bands. Currently the WMTS provides licensed bands in a 608 to 614 megahertz (MHz) range (also known as the 608 MHz band), a 1395 to 1400 MHz range (also known as the 1400 MHz band), and a 1427 to 1432 MHz range. The ISM bands include the popular 2.4 gigahertz (GHz) range (which currently includes frequencies from 2.4 to 2.5 GHz) and a 5 GHZ range (which currently includes frequencies from 5.725 to 5.875 GHz), which may be used by routers, wireless home telephones, or the like. Additionally, the Federal Communication Commission (FCC) in the United States is contemplating other potential bands such as a 3 GHz band for medical applications and a 2.3 GHz band for medical body area networks (MBANs). Other frequencies and frequency bands are set aside for use by specific companies or providers, such as cellular phone service providers. For example, wireless service providers may use licensed spectrums for communication with smartphones, tablets, wireless hotspots, or other mobile communication devices.

Note that the designation of frequencies or frequency bands for a specific purpose, whether licensed or unlicensed, may be under the control of a governmental body or standard setting organization. Thus, frequencies and frequency bands set aside for various purposes are subject to change over time and also can vary between different countries or geographic regions. For example, the FCC may eliminate, add, narrow, broaden, or create new licensed or designated bands. Furthermore, different countries may set aside different frequencies or frequency bands for medical, cellular, industrial, or other services. Although the present disclosure generally discusses embodiments in relation to licensed and/or designated frequencies within the United States, the present disclosure also contemplates and encompasses embodiments having modifications or variations for other countries or changes to designated frequency bands within a country.

The access radios 108 may relay or communicate data signals between the portable telemetry device 102 and the monitoring system 106. In one embodiment, an access radio 108 may communicate information directly to the monitoring system 106. For example, the access radio 108 may be located at a hospital campus or other medical facility and may communicate information to the monitoring system 106 directly or via a local area network (LAN). In one embodiment, an access radio 108 may communicate information to the monitoring system 106 via a network 110, such as a local area network (LAN), wide area network (WAN), cellular network, the Internet, and/or any other network. For example, a portable telemetry device 102 may be configured to communicate with the monitoring system 106 over a cellular network and the Internet. In one embodiment, a single portable telemetry device 102 may be configured for selective, or simultaneous, communication over one or more different access radios 108 using different frequencies or communication standards.

The monitoring system 106 may include a computing device such as a computer, server, or the like. The monitoring system 106 may include a processor, circuitry, computer readable memory, communication ports, and/or the like. In one embodiment, the monitoring system 106 may include a radio and/or an antenna. In one embodiment, one or more of the radio and the antenna may be separate from the monitoring system 106. For example, a radio or antenna may be connected to be in communication with the monitoring system 106 to relay information from a telemetry device to the monitoring system 106. In one embodiment, the monitoring system 106 includes a computing system for a central nurses station. For example, the monitoring system 106 may include a computing system for an intensive care ward, step down ward, or in-patient ward. Similarly, the monitoring system 106 may include a computing system for any monitoring system of a medical facility, such as a nurses station, monitoring war room, or the like. In one embodiment, the monitoring system 106 may include a portable monitoring system which is mobile and may be moved with a medical worker through a hospital or other medical facility.

The monitoring system 106 receives the physiological data from the portable telemetry device 102 and stores and/or processes the physiological data. In one embodiment, the monitoring system 106 stores the physiological data in memory for later access and/or analysis. In one embodiment, the monitoring system 106 processes the physiological data to detect problems for the patient, detect whether there is an alarm condition, or perform other analysis. For example, the monitoring system 106 may report an alarm condition to a nurse, doctor, or other medical personnel. The monitoring system 106 may also provide control data to the portable telemetry device 102 to configure alarm settings, reset alarms, determine a state or location of the portable telemetry device 102, transfer stored data, or configure operation of the portable telemetry device 102. In one embodiment, the monitoring system 106 may send and receive control data between the portable telemetry device 102 to determine that messages were received or that instructions corresponding to control data were performed.

In one embodiment, the monitoring system 106 is configured to provide two-way voice communication between medical staff located at the monitoring system 106 and a patient located with the portable telemetry device 102. For example, medical staff using the monitoring system 106 may be able to accept, initiate, end, or otherwise participate in a two-way communication with a patient or other individual located with a telemetry device 102. As an example, the two-way communication may operate similarly to a voice telephone call. The voice communication and physiological data provide a powerful combination of monitoring tools for medical staff.

As discussed above, the system 100 of FIG. 1 is given by way of example only. Numerous changes and variations with regard to the system 100 are considered within the scope of the present disclosure. For example, although FIG. 1 illustrates the access radios 108 as separate from the monitoring system 106, the monitoring system 106 may include one or more radios, antennas, or the like for wireless communication with telemetry devices 102. In one embodiment, the monitoring system 106 may be connected to an access point that includes an antenna only or includes both a radio and an antenna. For example, an antenna for receiving and sending signals may be connected to a radio that is part of the monitoring system 106. In one embodiment, both a radio and an antenna may be included as part of the monitoring system 106.

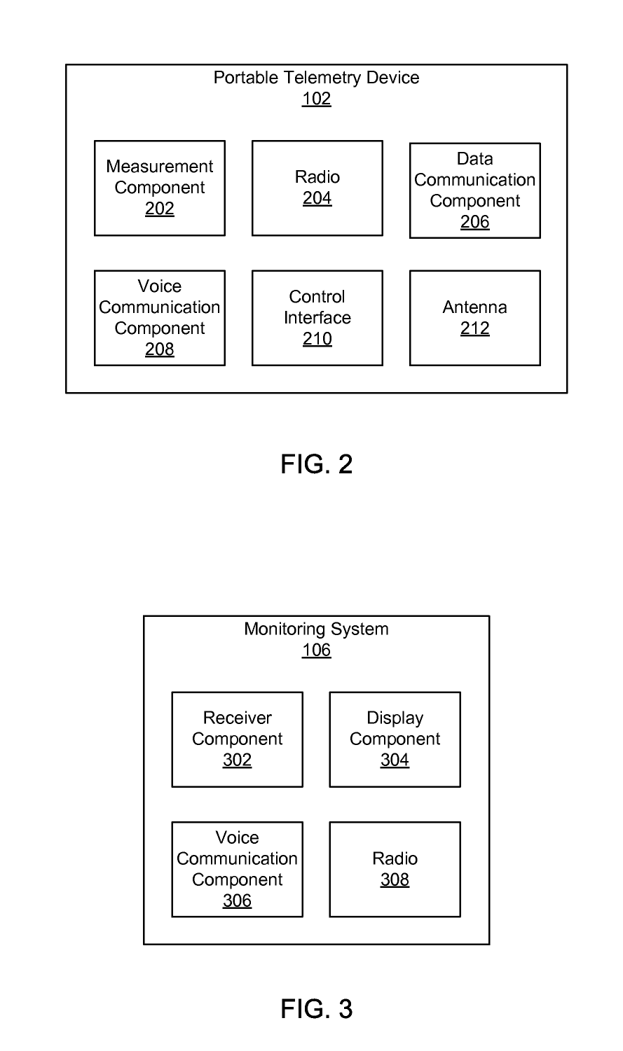

FIG. 2 is a schematic block diagram illustrating components of one embodiment of a portable telemetry device 102. The portable telemetry device 102 includes a measurement component 202, a radio 204, a data communication component 206, a voice communication component 208, a control interface 210, and an antenna 212. The components 202-212 are given by way of example only and may not all be included in all embodiments. In one embodiment, the portable telemetry device 102 may be used for patient monitoring within a hospital, ambulance, home, or other environment. The portable telemetry device 102 may include a patient-worn telemetry device that allows a patient to move while wearing the portable telemetry device and being monitored. For example, the portable telemetry device 102 may be clipped to a patient's clothing, or worn on a strap wrapped around the patient.

The measurement component 202 is configured to receive physiological data from one or more sensors. For example, the measurement component 202 may include one or more ports to connect to one or more sensor leads. Sensors may be attached to a patient and then attached to the portable telemetry device 102 via connection ports. With the portable telemetry device 102 worn by the patient, the measurement component 202 may receive the physiological data while still allowing the patient to ambulate or be moved around. The measurement component 202 may receive the physiological data in analog, digital, or other format. In one embodiment, the measurement component 202 receives the physiological data in an analog format and converts the data to a digital format for communication to a monitoring system 106.

The physiological data may include any type of data gathered by attached sensors 104. For example, the physiological data may include data regarding cardiac health, respirations, oxygen levels, or any other physiological condition of a patient. The measurement component 202 may process the physiological data to detect alarm states, convert from an analog to digital format, produce waveforms, calculate numerical data, or perform any other processing. In one embodiment, the measurement component 202 stores at least a portion of the physiological data.

The radio 204 includes a radio configured to wirelessly send and receive data signals. The radio 204 may include a radio configured to operate within a licensed or unlicensed frequency band. In one embodiment, the radio 204 may include off-the-shelf parts for communicating according to a wireless standard. In one embodiment, the radio 204 may be configured to operate according to an institute for electrical and electronics engineers (IEEE) 802.11 standard (known to industry groups as Wi-Fi), such as an 802.11 a, b, g, or n radio standard. In one embodiment, the radio 204 may be configured to operate within a licensed or restricted spectrum, such as within a spectrum defined by the WMTS and/or a spectrum licensed by a cellular communications provider. For example, the radio may implement a 3G, LTE, or any other cellular wireless communication standard. The radio 204 may include one, two, or more wireless radios configured to operate within different wireless frequencies and/or according to different communication standards.

A coverage area for the radio 204 may depend, at least in part, on the frequencies and standards for which the radio 204 is capable of implementing. For example, a radio 204 configured to operate within a restricted medical spectrum may provide a coverage area approximating a medical facility campus. A radio 204 configured to operate within an ISM band, or other unlicensed band, may not provide significant coverage but may be able to provide coverage at different specific locations due to the wide availability of Wi-Fi networks. A radio 204 configured to operate within a licensed cellular band may provide the widest outdoor coverage area due to the potential general availability of mobile network signals within a large coverage area. According to one embodiment, once the radio 204 gets outside of its coverage area, the portable telemetry device 102 may no longer be able to communicate with a monitoring system 106. In one embodiment, the standards and frequencies implemented by the radio 204 may be selected for a desired coverage area and/or dependability for the portable telemetry device 102.

The antenna 212 may include an antenna for use with a desired communication frequency or standard. For example, the antenna 212 may be a Wi-Fi, Bluetooth, or other antenna which the radio 204 can use to send and receive wireless signals. Varying embodiments may include the antenna 212 as either an internal or external antenna.

The data communication component 206 is configured to communicate physiological data to a monitoring system 106. For example, the data communication component 206 may send physiological data received and/or processed by the measurement component 202 to the monitoring system 106 using the radio 204. The data communication component 206 may send the physiological data that includes waveform data, numerical data, or any other type of physiological data. Similarly, the data communication component 206 may also communicate other information about the physiological data, attached sensors, or a status of the portable telemetry device 102. For example, the data communication component 206 may use the radio 204 to send or receive one or more of alarm limit data, alarm reset data, configuration data for the portable telemetry device 102, and stored patient data. The alarm limit data may include data that defines limits, which, when exceeded or fallen below, will trigger an alarm. The portable telemetry device 102 may configure alarm settings based on the alarm limit data. The alarm reset data may include data that indicates that an alarm should be reset. For example, after an alarm is triggered, a nurse or other medical personnel may check on the patient. The nurse may cause an alarm reset signal to be sent to a monitoring system 106 to indicate that an issue is being addressed or that an issue has been resolved. The configuration data may indicate what physiological data to report, how frequently it should be reported, or the like. In one embodiment, the configuration data may include a battery level of the portable telemetry device 102 or otherwise indicate whether the portable telemetry device 102 is operating correctly.

The voice communication component 208 allows the portable telemetry device 102 to be used as a two-way communication device. For example, the voice communication component 208 may be configured to provide, using the wireless radio 204, two-way voice communication between the portable telemetry device 102 and medical staff at a monitoring system 106. In one embodiment, the voice communication component 208 may be configured to provide two-way communication similar to a two-way radio. For example, communication may be controlled based on holding down a button to speak and releasing the button to listen.

In one embodiment, the voice communication component 208 may be configured to provide session-based two-way communication, similar to a telephone or telephone system. In one embodiment, the voice communication component 208 provides two-way voice communication using voice over internet protocol (VoIP). VoIP includes methods and technologies for providing voice communications over an Internet protocol (IP) network, such as a LAN, WAN, or the Internet. The voice communication component 208 may initiate, accept, maintain, and/or end a voice session based on the VoIP or any other protocol. In one embodiment, the voice communication component 208 may accept incoming requests for a two-way voice communication session from the monitoring system 106 without requiring input from a user. For example, if a monitoring system 106 is designated as corresponding to the portable telemetry device 102, the voice communication component 208 may accept and initialize a two-way communication session if requested by the monitoring system 106. In other embodiments, the voice communication component 208 may be configured to only accept or send a request for voice communication in response to input by a user at the portable telemetry device 102.

The control interface 210 may allow a user located at the portable telemetry device 102 to control operation of the portable telemetry device 102 and/or control two-way communication. In one embodiment, the control interface 210 may provide physical buttons or a screen that displays buttons or icons that can be selected by a patient or other individual at the location of the portable telemetry device 102 to control two-way voice communication. For example, the voice communication component 208 may send or accept a request to begin the two-way voice communication in response to input from a user via the control interface 210. Similarly, the voice communication component 208 may end the two-way voice communication in response to input from a user via the control interface 210.

Telephone type communication may allow a monitoring system 106 to selectively communicate with only one or more portable telemetry devices 102 at a time. For example, a session with a specific telemetry device 102 may be begun, maintained, and ended independently of communication with other telemetry devices 102. In one embodiment, the voice communication component 208 of the portable telemetry device 102 may be configured to initiate or maintain a session with a specific monitoring system. In one embodiment, if a call is not answered, the call may ring through to a backup monitoring system.

The control interface 210 may also be used to control other aspects of the operation of the portable telemetry device 102. For example, a user (such as a nurse) may be able to view physiological data on the portable telemetry device 102, power the portable telemetry device 102 on or off, or determine or change current settings for the portable telemetry device 102. In one embodiment, the control interface 210 may restrict certain functions for authorized individuals. For example, only authorized individuals, as determined based on a passcode or any other credentials, may be allowed to make changes to alarm settings, a powered state, or other operations. In one embodiment, the control interface 210 may provide a simple single button interface for initializing, accepting, or ending two-way communication with a monitoring system 106.

The portable telemetry device 102 may also include a variety of other components. For example, the portable telemetry device 102 may include a built-in microphone, speaker, or the like to allow a patient or other individual present with the portable telemetry device 102 to carry on a hands-free two-way conversation with medical staff at a monitoring system 106.

FIG. 3 is a schematic block diagram illustrating components of one embodiment of a monitoring system 106. The monitoring system 106 includes a receiver component 302, a display component 304, a voice communication component 306, and radio 308. In one embodiment, the monitoring system 106 is configured to communicate with a plurality of portable telemetry devices 102. The components 302-308 are given by way of example only and may not all be included in all embodiments.

The receiver component 302 is configured to receive physiological data from one or more portable telemetry devices 102. For example, the receiver component 302 may receive the physiological data sent by a data communication component 206 of a portable telemetry device 102. The receiver component may also receive information regarding alarms, alarm limits, or a status of the portable telemetry device 102. In one embodiment, the receiver component 302 receives the physiological data via an access radio 108 and/or a network interface card (NIC). For example, the access radio 108 may be in communication with, or part of, the monitoring system 106. The access radio 108 may receive signals corresponding to the physiological data and forward the physiological data to the monitoring system 106. In one embodiment, the receiver component 302 receives the physiological data via NIC. For example, the monitoring system 106 may include a NIC to allow the connection or communication with a communications network.

In one embodiment, the receiver component 302 may store the received data for later processing or may forward the data onto a processor or another component 304-306 for processing. When physiological data is received, the receiver component 302 may identify a patient that corresponds to the data and store the physiological data in a database or location corresponding to the patient. For example, the physiological data may be transmitted with a patient identifier. The receiver component 302 may look up the identifier to determine where the physiological data should be stored. In one embodiment, a plurality of different patients within a hospital or other medical center may be wearing portable telemetry devices 102. The receiver component 302 may store physiological data received from each patient-worn portable telemetry device 102 separately to maintain the data separately and/or securely.

The display component 304 is configured to display information corresponding to the physiological data, or other data, received by the receiver component 302. For example, the display component 304 may provide a monitoring interface that displays patient numerical or waveform data illustrating the physiological data on a display. Additionally, information about alarm settings and/or any received alarms may also be displayed. In one embodiment, the display component 304 may display physiological data for only a single patient at a time. In one embodiment, the display component 304 may display physiological data for two or more or all of the patients that correspond to the monitoring system 106. For example, the monitoring system 106 may be assigned one or more portable telemetry devices 102 (or corresponding patients) to monitor.

One or more medical staff can view the physiological data, monitoring settings, and/or alarms to monitor the status of a telemetry system 100 and/or the health of one or more patients. In one embodiment, the display component 304 displays physiological data for a patient that corresponds to a current two-way communication session monitoring system. For example, if a user presses a call button on the portable telemetry device 102 to initiate a communication session, the monitoring system 106 may initiate communication and switch to a display of the patient data for the corresponding patient. The medical staff may thereby be able to quickly examine the physiological data and coordinate the data, if any, with any symptoms reported by the patient.

The voice communication component 306, similar to the voice communication component 208 of FIG. 2, is configured to provide two-way voice communication with a portable telemetry device 102. For example, the voice communication component 306 may be configured to provide, using the access radios 108, two-way voice communication between a portable telemetry device 102 and medical staff at a monitoring system 106.

In one embodiment, the voice communication component 306 provides session-based two-way communication, similar to a telephone or telephone system. In one embodiment, the voice communication component 306 provides two-way voice communication using voice over internet protocol (VoIP). VoIP includes methods and technologies for providing voice communications over an Internet protocol (IP) network, such as a LAN, WAN, or the Internet. The voice communication component 306 may initiate, accept, maintain, and/or end a voice session based on the VoIP or any other protocol. In one embodiment, the voice communication component 306 may initiate two-way voice communication with a portable computing device 102 without requiring input at the portable telemetry device 102 end. For example, medical staff can initiate communication even in the event that the patient is not responding or does not accept a call.

In one embodiment, the display component 304 may provide a control interface to control the beginning or end of a communication session. Similarly, a physical interface (such as a conventional telephone and/or telephone interface) may be used to control voice communication. For example, the voice communication component 306 may send or accept a request to begin the two-way voice communication in response to input from a user via the control interface 210. In one embodiment, medical staff may enter a number using a keypad, such as a telephone number, patient number, room number, or the like, to begin a call with a specific portable telemetry device 102. Alternatively, icons corresponding to a portable telemetry device 102 may be displayed on a display and selected by a nurse or other medical staff to begin voice communication. Similarly, the voice communication component 306 may end the two-way voice communication in response to input from a user via the control interface 210. For example, medical staff may be able to hang up the phone or press a physical button to end the call. In one embodiment, icons or the like are displayed on a display screen which may be selected to end the voice communication.

As discussed above, telephone-type communication may enable medical staff at a monitoring system 106 to selectively communicate with only one or more portable telemetry devices 102 at a time. For example, a session with a specific telemetry device 102 may be begun, maintained, and ended independently of communication with other telemetry devices 102. In one embodiment, the voice communication component 306 of the portable telemetry device 102 may be configured to initiate or maintain a session with a specific monitoring system 106.

The radio 308 may include a radio that is configured to send and receive data on behalf of other components 302-306 of the monitoring system 106. In one embodiment, the radio 308 may include an antenna or be in communication with an antenna. For example, an antenna separate from the monitoring system 106 may be connected to the radio 308 to enable wireless communication. In some embodiments, the monitoring system 106 may be in communication with a radio 308 separate from the monitoring system 106.

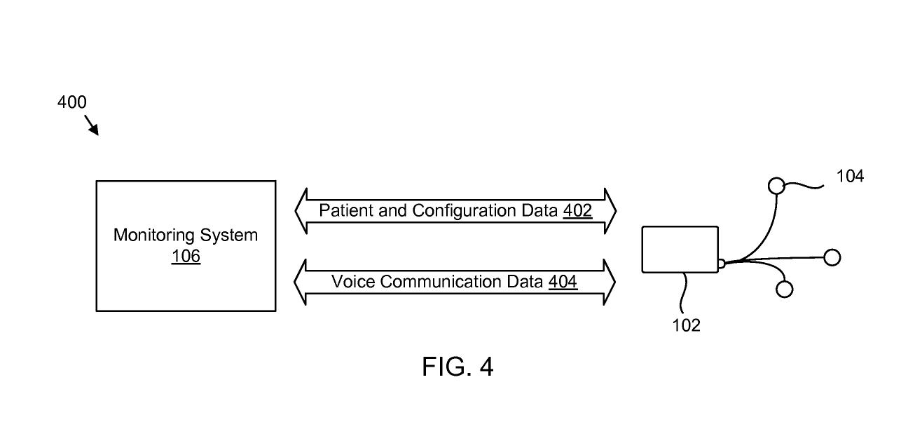

FIG. 4 is a schematic diagram illustrating communication of different types of data between a portable telemetry device 102 and a monitoring system 106. The data communicated between the monitoring system 106 and telemetry device 102 may include patient and configuration data 402 and voice communication data 404. The patient and configuration data 402 may include physiological parameters, patient identification information, monitoring limits, configuration data, or alarm data. For example, the patient and configuration data 402 may include data to monitor the physiological parameters of the patient and may include any non-voice data discussed herein. The voice communication data 404 may include data corresponding to voice communications between the portable telemetry device 102 and the monitoring system 106. For example, audio data and control data for a VoIP session may be communicated as voice communication data 404.

In one embodiment, the patient and configuration data 402 is repeatedly or continually sent during monitoring. For example, the patient and configuration data 402 may be sent as long as a corresponding patient is to be monitored by the monitoring system 106. Thus, an active connection for the patient and configuration data 402 may be maintained as continuously as possible. However, at least a portion of the voice communication data 404 may only need to be communicated during an active call. For example, a call may only be infrequently active between the monitoring system 106 and the portable telemetry device 102. Specifically, voice communication may only be active after a patient or medical worker initiates the call and may be ended after a short conversation.

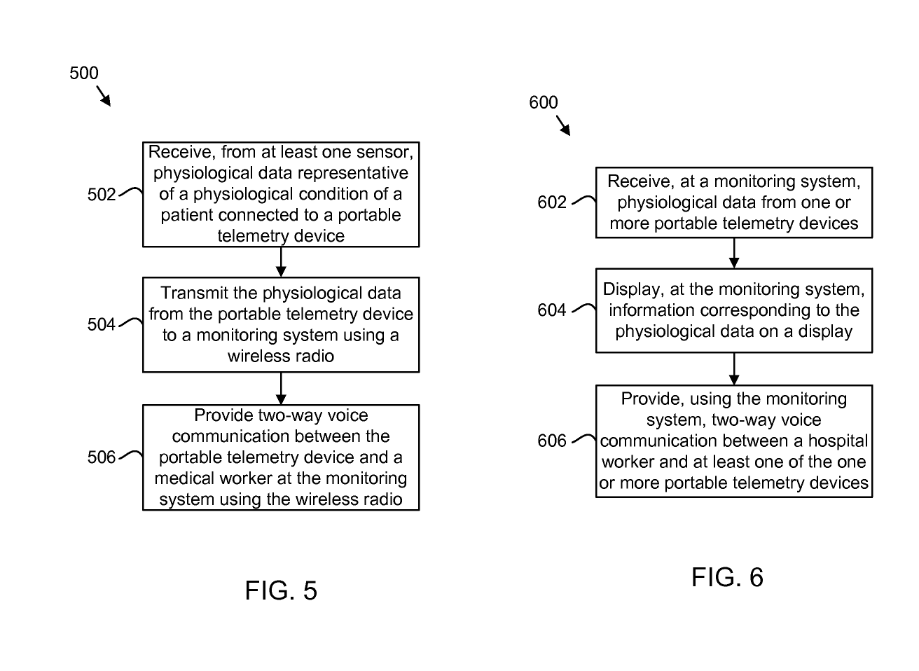

FIG. 5 is a schematic flow chart diagram illustrating a method 500 for wireless telemetry, according to one embodiment. The method 500 may be performed by a portable telemetry device 102, such as the portable telemetry device 102 of FIG. 2.

The method 500 begins and a measurement component 202 receives 502 physiological data from one or more sensors 104. The one or more sensors 104 may be connected to a patient corresponding to the portable telemetry device 102. For example, the portable telemetry device 102 may be worn or carried by the patient. The measurement component 202 may receive 502 the physiological data via one or more sensor leads plugged into a port of the portable telemetry device 102. The physiological data from the sensors 104 may indicate a physiological condition of a patient. For example, the physiological data may include data regarding cardiac health, respirations, oxygen levels, or any other physiological condition of a patient.

A data communication component 206 transmits 504 the physiological data from the portable telemetry device to a monitoring system using a radio 204. In one embodiment, the data communication component 206 transmits 504 the physiological data to an access radio 108 using a radio 204 configured to operate on a licensed spectrum, such as WMTS band or a licensed cellular band. In one embodiment, the data communication component 206 transmits 504 the physiological data to an access radio 108 using a radio 204 configured to operate in an unlicensed spectrum, such as an ISM band. The access radios 108 may provide the physiological data to the monitoring system 106.

A voice communication component 208 provides 506 two-way voice communication between the portable telemetry device 102 and a medical worker at the monitoring system 106 using the radio 204. For example, the voice communication component 208 may communicate with the monitoring system 106 using a VoIP protocol. Thus, a patient wearing a portable telemetry device 102, or another nearby individual, may be able to talk with medical staff as long as the patient and/or portable telemetry device 102 are within range of an access radio 108.

FIG. 6 is a schematic flow chart diagram illustrating another method 600 for wireless telemetry, according to one embodiment. The method 600 may be performed by a monitoring system 106, such as the monitoring system 106 of FIG. 3.

The method 600 begins and a receiver component 302 receives 602 physiological data from one or more portable telemetry devices 102. For example, the physiological data may correspond to attached patients and may indicate a physiological condition of each patient. In one embodiment, the physiological data is transmitted with an identifier identifying the corresponding patient.

A display component 304 displays 604 information corresponding to the physiological data on a display. For example, the display component 304 may display 604 waveforms, numerical data, alarms, or the like at a central monitoring location. For example, the central monitoring location may include a central nurses station or a monitoring war room for a medical facility. In one embodiment, the display component 304 displays 604 the physiological data on a mobile display.

A voice communication component 306 provides 606 two-way voice communication between a hospital worker and at least one of the one or more portable telemetry devices 102. For example, the hospital worker may be located with a monitoring system 106 and the voice communication component 306 provides 606 two-way voice communication between the portable telemetry device 102 and the monitoring system 106. In one embodiment, the voice communication component 306 provides 606 two-way voice communication simultaneously to the display component 304 displaying 604 the physiological data. In one embodiment, the voice communication component 306 provides 606 two-way voice communication in response to user input or to a request from the portable telemetry device 102.

Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium, wherein when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present invention. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

It will be obvious to those having skill in the art that many changes may be made to the details of the above-described embodiments without departing from the underlying principles of the invention. The scope of the present invention should, therefore, be determined only by the following claims.

1. A portable telemetry device comprising:
a measurement component configured to receive, from at least one sensor, physiological data representative of a physiological condition of a patient;
a wireless radio configured to wirelessly send and receive radio signals;
a data communication component configured to transmit the physiological data to a monitoring system using the wireless radio; and
a voice communication component configured to provide, using the wireless radio, two-way voice communication between the portable telemetry device and medical staff at the monitoring system.

2. The portable telemetry device of claim 1, wherein the voice communication component is configured to provide two-way voice communication using voice over internet protocol (VoIP).

3. The portable telemetry device of claim 1, further comprising a control interface to receive input from a user.

4. The portable telemetry device of claim 3, wherein the voice communication component is configured to, in response to the input, send or accept a request to begin the two-way voice communication.

5. The portable telemetry device of claim 3, wherein the voice communication component is configured to, in response to the input, end the two-way voice communication.

6. The portable telemetry device of claim 1, wherein the voice communication component is configured to begin the two-way voice communication in response to a request from the medical staff, wherein no input at the portable telemetry device is required to begin the two-way voice communication.

7. The portable telemetry device of claim 1, wherein the wireless radio is configured to send and receive radio signals within a licensed wireless spectrum.

8. The portable telemetry device of claim 7, wherein the licensed wireless spectrum corresponds to a cellular network, and wherein the wireless radio is further configured to wirelessly send and receive radio signals based on a cellular communication standard.

9. The portable telemetry device of claim 1, wherein the monitoring system comprises a central monitoring system of a medical facility.

10. The portable telemetry device of claim 1, comprising a patient-worn telemetry device.

11. A method for medical telemetry, the method comprising:
receiving, from at least one sensor, physiological data representative of a physiological condition of a patient connected to a portable telemetry device;
transmitting the physiological data from the portable telemetry device to a monitoring system using a wireless radio; and
providing two-way voice communication between the portable telemetry device and a medical worker at the monitoring system using the wireless radio.

12. The method of claim 11, wherein providing the two-way voice communication comprises providing the two-way voice communication using voice over internet protocol (VoIP).

13. The method of claim 11, further comprising receiving input from a user via a control interface of the portable telemetry device.

14. The method of claim 13, further comprising sending or accepting a request to begin the two-way voice communication in response to the input from the user.

15. The method of claim 13, further comprising ending the two-way voice communication in response to the input from the user.

16. The method of claim 11, wherein providing the two-way voice communication comprises beginning the two-way voice communication in response to a request from the medical staff, wherein input from a user at the portable telemetry device is not required for beginning the two-way voice communication.

17. The method of claim 11, wherein transmitting one or more of the physiological data and the voice communication comprises transmitting within a licensed wireless spectrum.

18. The method of claim 17, wherein the licensed wireless spectrum corresponds to a cellular network, and wherein the wireless radio is further configured to wirelessly send and receive radio signals based on a cellular communication standard.

19. A monitoring system comprising:
a receiver component configured to receive physiological data from one or more portable telemetry devices;
a display component configured to display information corresponding to the physiological data; and
a voice communication component configured to provide two-way voice communication between a hospital worker and at least one of the one or more portable telemetry devices.

20. The system of claim 19, wherein the voice communication component is configured to provide two-way voice communication using voice over internet protocol (VoIP).

21. The system of claim 19, wherein the display component is configured to display physiological data corresponding to at least one of the one or more portable telemetry devices during the two-way voice communication with at least one of the one or more portable telemetry devices.

22. The system of claim 19, further comprising one or more wireless radios configured to wirelessly communicate with the one or more portable telemetry devices.

23. The system of claim 22, wherein the one or more wireless radios are configured to send and receive radio signals within a licensed wireless spectrum.

24. The system of claim 23, wherein the licensed wireless spectrum corresponds to a licensed medical frequency band.

25. The system of claim 19, further comprising a monitoring control interface to receive input from a user.

26. The system of claim 25, wherein the voice communication component is configured to, in response to the input, send or accept a request to begin the two-way voice communication.

27. The system of claim 25, wherein the voice communication component is configured to, in response to the input, end the two-way voice communication.
